(19) United States
(12) Patent Application Publication
(10) Pub. No.: US 2025/0110099 A1
(43) Pub. Date: Apr. 3, 2025

(54) MEMBRANE-BIOCHAR STRIP FOR WATER SAMPLING

(71) Applicant: KING FAHD UNIVERSITY OF PETROLEUM AND MINERALS, Dhahran (SA)

(72) Inventors: Mazen Khaled NAZAL, Dhahran (SA); Nabeel Saeed Fuad ABUZAID, Dhahran (SA); Durga Rao GIJJAPU, Dhahran (SA)

(73) Assignee: KING FAHD UNIVERSITY OF PETROLEUM AND MINERALS, Dhahran (SA)

(21) Appl. No.: 18/480,165

(22) Filed: Oct. 3, 2023

(51) Int. Cl. G01N 33/18; C01B 32/318

(52) U.S. Cl. CPC G01N 33/1826; C01B 32/318; G01N 2333/415

(57) ABSTRACT

A membrane-biochar strip for water sampling includes two porous polymeric membrane sheets having the same dimension. The two porous polymeric membrane sheets have an average pore size of about 50 to 150 micrometers (μm). Seagrass biochar (SGBC) particles are disposed between two porous polymeric membrane sheets resulting in a sandwich structure. An edge of the sandwich structure is sealed, and the SGBC particles are encapsulated in the membrane-biochar strip. The SGBC particles have an average particle size of about 150 to 400 μm. The seagrass biochar (SGBC) particles are prepared from Halodule Uninervis seagrass.

### STATEMENT OF ACKNOWLEDGEMENT

This research was supported by the Applied Research Center for Environment and Marine Studies, at King Fahd University of Petroleum and Minerals Dhahran, Saudi Arabia.

### BACKGROUND

### Technical Field

The present disclosure is directed to a biochar adsorbent, and more particularly to a membrane-biochar strip for water sampling.

### DESCRIPTION OF THE RELATED PRIOR ART

The description of the related prior art provided herein is for the purpose of generally presenting the context of the disclosure. Work of the presently named inventors, to the extent described in this background section, as well as aspects of the description which may not otherwise qualify as prior art at the time of filing, are neither expressly nor impliedly admitted as prior art against the present invention.

Water pollution poses risks to aquatic life, including fish and fish-consuming wildlife as well as to human health. Therefore, monitoring water contamination to protect ecosystems and public health is imperative. As such, the need for measuring contaminant concentration has led to the development of water sampling methodologies for determining dissolved contaminant masses in water. Known samplers often involve a sampler enclosure having an adsorbent disposed therein. The sampler is positioned within a water body, held at a certain location by one or more supports, such as cables, buoys, or rigid arms. Particularly, the sampler is placed strategically in water to have the fluid come into contact with the adsorbent, thereby adsorbing the pollutants. If the water comes in contact with the adsorbent under natural fluid forces, then the process is a passive sampling process. If the water is forced into the adsorbent of the sampler by pumping, then the process is an active sampling process. Samplers are retrieved after a predetermined period in both sampling processes and analyzed in the laboratory to measure ion exchange and characterize the nature and concentration of pollutants present in the collected samples. Thus, an environment specialist can establish baseline concentrations from the collected pollutants, determine locations of polluting sources, and identify pollutant migration and distribution routes.

Generally, samplers play a pivotal role in mitigating potential harm to the environment and public health. They serve a dual purpose by facilitating both rapid, short-term environmental assessments and sustained, long-term monitoring to prevent catastrophic environmental events. Both active and passive sampling processes are vital in achieving these goals. The active sampling process has certain advantages, such as a faster sampling rate and the ability to capture larger quantities of pollutants compared to the passive sampling method. Further, the active sampling process is more efficient than the passive sampling process, albeit at a higher cost than the passive sampling process. However, the active sampling process involves energy consumption for getting contaminants adsorbed onto the adsorbent of the sampler.

Some of the most commonly used passive samplers in environmental monitoring include but are not limited to Polar Organic Chemical Integrative Samplers (POCIS) samplers, semipermeable membrane devices (SPMDs), polymer sheets, ceramic dosimeters, diffusive gradients in thin-film (DGT), discrete multilayer samplers (DMLS), membrane-enclosed sorptive coating (MESCO) samplers, passive integrative mercury samplers (PIMS), passive in situ concentration-extraction samplers (PISCES), and Chemcatcher or passive organic chemical intumescence samplers. Among them, POCIS samplers emerge as an option due to their cost-effectiveness, absence of power dependencies, and minimal maintenance needs for the isolation and concentration of low-level organic compounds within aquatic environments. POCIS can function as both kinetic and equilibrium samplers, comprising two primary parts: adsorbent and polymeric membrances, e.g., polyether sulfone (PES) membranes.

Commonly used adsorbents include materials such as 3M, Empore™, chelating disks, C18 disks, Strata-X, Oasis MAX, Chromabond HRX, Strata XAW, Sepra ZT, Oasis WAX, Strata X-CW, ionic liquids, Strata XAW mixed with Bond-Elute Plexa, molecularly imprinted polymers, and carbon nanotube sorbents. Each one of these materials has its advantages and disadvantages. Some of them have relatively high adsorption efficiency and satisfactory sampling rates, e.g., C18 disks, ionic liquids, molecularly imprinted polymers, and carbon nanotube sorbents. However, they generally come with a relatively high cost, carry some toxicity concerns, and require complex multistep synthesis processes. Therefore, there is a crucial need to develop adsorbents that combine the essential uptake efficiency with the cost-effectiveness of readily available raw materials.

Although literature reveals various adsorbents, however, there is a need for a strategy to develop a simple and efficient adsorbent that may eliminate or overcome the aforementioned limitations.

In view of the foregoing, it is an objective of the present disclosure to provide a membrane-biochar strip for water sampling. A second objective of the present disclosure is to provide a water sampling method for the determination of a dissolved contaminant in an aqueous liquid, the method.

### SUMMARY

In an exemplary embodiment, a membrane-biochar strip for water sampling is described. The membrane-biochar strip includes two porous polymeric membrane sheets having the same dimension. In some embodiments, the two porous polymeric membrane sheets have an average pore size of about 50 to 150 micrometers (μm). In some embodiments, seagrass biochar (SGBC) particles are disposed between two porous polymeric membrane sheets resulting in a sandwich structure. In some embodiments, an edge of the sandwich structure is sealed, and the SGBC particles are encapsulated in the membrane-biochar strip. In some embodiments, the SGBC particles have an average particle size of about 150 to 400 μm. In some embodiments, the seagrass biochar (SGBC) particles are prepared from Halodule Uninervis seagrass.

In some embodiments, each of the two porous polymeric membrane sheets are independently selected from the group consisting of a polypropylene (PP) membrane sheet, a polyethersulfone (PES) membrane sheet, and a low-density polyethylene (LDPE) membrane sheet.

In some embodiments, the two porous polymeric membrane sheets are polypropylene (PP) membrane sheets.

In some embodiments, the two porous polymeric membrane sheets have an average pore size of about 100 μm.

In some embodiments, the SGBC particles have an average particle size of about 250 μm. In some embodiments, the SGBC particles have a porous structure, including a plurality of fractures and grooves. In some embodiments, the SGBC particles have a long dimension of 250 μm. In some embodiments, the SGBC particles have a thickness of 0.5 to 5 μm. In some embodiments, the SGBC particles have a width of 5 to 25 μm.

In some embodiments, the SGBC particles have a surface area of 50 to 70 square meters per gram (m²/g).

In some embodiments, the SGBC particles have a pore volume of 0.05 to 0.1 cubic centimeters per gram (cm³/g).

In some embodiments, the SGBC particles have an absorption capacity of up to 20 milligrams (mg) of a phenolic compound per gram of the SGBC particles.

In some embodiments, the SGBC particles includes 58 to 68 wt. % C, 10 to 15 wt. % 0, 5 to 15 wt. % K, 2 to 12 wt. % Cl, 1 to 5 wt. % Ca, 1 to 5 wt. % S, 0.1 to 2 wt. % Mg, each wt. % based on a total weight of the SGBC particles, as determined by energy dispersive X-ray (EDX) analysis.

In some embodiments, the SGBC particles includes about 63.7 wt. % C, about 12.3 wt. % O, about 10.6 wt. % K, about 7.8 wt. % Cl, about 2.4 wt. % Ca, about 2.2 wt. % S, about 1.0 wt. % Mg, each wt. % based on the total weight of the SGBC particles, as determined by energy dispersive X-ray (EDX) analysis.

In another exemplary embodiment, a water sampling method for the determination of a dissolved contaminant in an aqueous liquid is described. The method includes immersing the membrane-biochar strip in the aqueous liquid including the dissolved contaminant thereby allowing molecules of the dissolved contaminant to pass through the two porous polymeric membrane sheets of the membrane-biochar strip and contact with the SGBC particles. The method includes removing the membrane-biochar strip from the aqueous liquid after the immersing and extracting the dissolved contaminant from the membrane-biochar strip with an organic solvent to produce an extraction solution including the dissolved contaminant molecules.

In some embodiments, the dissolved contaminant is present in the aqueous liquid at a concentration of 1 to 20 milligrams per liter (mg/L) of the aqueous liquid.

In some embodiments, the method has a sampling rates (Rₛ) in a range of 0.005 to 0.045 liter per day (L/day) per 100 milligrams (mg) of the SGBC particles encapsulated in the membrane-biochar strip.

In some embodiments, the dissolved contaminant is at least one phenolic compound selected from the group consisting of phenol, 2-chlorophenol (2-CP), 2-methyl phenol (2-MP), 2,4-dimethyl phenol (2,4-DMP), 2,4-dichlorophenol (2,4-DCP), and 2-nitro phenol (2-NP).

In some embodiments, the organic solvent is at least one selected from the group consisting of benzene, cyclohexane, ethanol, methanol, acetone, ethyl acetate, dichloromethane, toluene, and diethyl ether.

In some embodiments, the method further includes directly injecting the extraction solution including the dissolved contaminant molecules into a mass spectrometer or a chromatography column for the determination of the dissolved contaminant.

In some embodiments, the method further includes preparing the SGBC particles by washing raw seagrass and heating at a temperature of about 500 degrees Celsius (° C.) in an atmosphere of an inert gas to form the SGBC in the form of particles.

In some embodiments, the raw seagrass includes Zostera Marina seagrass, Posidonia Oceanica seagrass, Halodule Uninervis seagrass, Thalassia Testudinum seagrass, Syringodium Filiforme seagrass, Enhalus Acoroides seagrass, Cymodocea Nodosa seagrass, Thalassodendron Ciliatum seagrass, Phyllospadix Spp seagrass, and Posidonia Australis seagrass.

In some embodiments, the raw seagrass is Halodule Uninervis seagrass.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic flow chart of a method for the determination of a dissolved contaminant in an aqueous liquid, according to certain embodiments;

FIG. 1B is a scanning electron microscopy (SEM) image depicting surface morphology of the prepared biochar at magnification 300×, according to certain embodiments;

FIG. 2 depicts an energy dispersive X-ray (EDX) spectrum of seagrass biochar (SGBC), according to certain embodiments;

FIG. 3 depicts a Fourier transform infrared spectroscopy (FTIR) spectrum showing the main functional groups onto the surface of the SGBC, according to certain embodiments;

FIG. 4 is as schematic illustration depicting fabrication of a sampling strip including a membrane packed with the biochar derived from seagrass or SGBC, according to certain embodiments; and

FIG. 5A is a graph depicting mass of phenol bound on the developed sampling strips vs. sampling time, according to certain embodiments;

FIG. 5B is a graph depicting mass of 2-chlorophenol (CP) bound on the developed sampling strips vs. sampling time, according to certain embodiments;

FIG. 5C is a graph depicting mass of 2-methylphenol (MP) bound on the developed sampling strips vs. sampling time, according to certain embodiments;

FIG. 5D is a graph depicting mass of 2,4-dimethylphenol (2,4-DMP) bound on the developed sampling strips vs. sampling time, according to certain embodiments;

FIG. 5E is a graph depicting mass of 2,4 dichlorophenol (2,4-DCP) bound on the developed sampling strips vs. sampling time, according to certain embodiments; and

FIG. 5F is a graph depicting mass of 2-nitrophenol (NP) bound on the developed sampling strips vs. sampling time, according to certain embodiments.

### DETAILED DESCRIPTION

When describing the present disclosure, the terms used are to be construed in accordance with the following definitions, unless a context dictates otherwise. Embodiments of the present invention will now be described more fully hereinafter with reference to the accompanying drawings wherever applicable, in which some, but not all embodiments of the disclosure are shown.

Further, as used herein, the use of singular includes plural and the words “a”, “an” includes “one” and means “at least one” unless otherwise stated in this application.

As used herein, the words “about,” “approximately,” or “substantially similar” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

The use of the terms “include,” “includes”, “including,” “have,” “has,” or “having” should be generally understood as open-ended and non-limiting unless specifically stated otherwise.

As used herein, the term “membrane,” or “porous polymeric membrane sheets” as used herein refers to a porous structure that is capable of separating components of a homogeneous or heterogeneous fluid with a solid. In particular, “pores” in the sense of the present disclosure indicate voids allowing fluid communication between different sides of the structure. More particularly, in use when a homogeneous or heterogeneous fluid is passed through the membrane, some components of the fluid can pass through the pores of the membrane into a “permeate stream”, some components of the fluid can be retained by the membrane and can thus accumulate in a “retentate” and/or some components of the fluid can be rejected by the membrane into a “rejection stream”. Membranes can be of various thicknesses, with homogeneous or heterogeneous structures. Membranes can be in the form of flat sheets or bundles of hollow fibers.

Membranes can also be in various configurations, including but not limited to spiral wound, tubular, hollow fiber, and other configurations identifiable to a skilled person upon a reading of the present disclosure. Membranes can also be classified according to their pore diameter. Membranes can be neutral or charged, and particle transport can be active or passive. The latter can be facilitated by pressure, concentration, and chemical or electrical gradients of the membrane process. In the present disclosure, the “membrane” is not a sorbent for the purposes of the present disclosure.

Aspects of the present disclosure are directed toward an efficient adsorbent/sampling device, achieved by the conversion of seagrass into biochar, which is subsequently transformed into a sampling strip. Biochar is a resource-rich material featuring carbon and functional groups as well as its cost-effectiveness due to the ready availability of its raw materials. Its production process involves relatively low temperatures, thereby addressing the limitations of previous approaches in this field.

According to an aspect of the present disclosure, a membrane-biochar strip for water sampling is described. The membrane-biochar strip includes two porous polymeric membrane sheets having the same dimension. In some embodiments, the two porous polymeric membrane sheets may be in the form of a porous membrane bag without additional membranous layers, coatings, films, filtration assemblies, holders, or other external components since a porous membrane bag having a single layer of the porous polymeric membrane sheet provides more rapid and complete perfusion of an aqueous liquid. Thus, for use, it may be unnecessary to insert the two porous polymeric membrane sheets in the form of a porous membrane bag into a frame, carrier, filter cartridge assembly, or other mechanical device to affix a filter. However, in some alternative embodiments, two, three, or more porous polymeric membrane sheets may be used to form a porous membrane bag or the porous membrane bag may be incorporated as part of a larger system comprising other elements such as filters, holders, or other external components.

In one embodiment, the membrane-biochar strip comprising the two porous polymeric membrane sheets has a shape that is triangular, tetrahedral, square, cubic, rectangular, parallelepiped, circular, spherical, pouch-like, sachet-like, purse-like, or other shape that prevents release of seagrass biochar (SGBC) particles from the membrane-biochar strip. In some embodiments, two or more porous polymeric membrane sheets are sealed together to produce the membrane-biochar strip in the form of a porous membrane bag, for example, two rectangular or square-shaped porous polymeric membrane sheets may have the SGBC particles placed between their surfaces resulting in a sandwich structure and then an edge of the sandwich structure is sealed and the SGBC particles are encapsulated in the sandwich structure to form the membrane-biochar strip. In another embodiment, two circular or oval-shaped porous polymeric membrane sheets may be loaded with the SGBC particles and sealed to form a circular or oval-shaped pod. In other embodiments, only a single porous polymeric membrane sheet will be used to produce a membrane-biochar strip enclosing the SGBC particles. Typically, the membrane-biochar strip permanently seals in the SGBC particles so as to prevent its accidental escape into the bulk of the aqueous liquid, and/or organic solvent.

In some embodiments, the membrane-biochar strip comprises pleats or folds that permit the membrane-biochar strip to open or expand after contact with an aqueous liquid containing a dissolved contaminant, or when holding a sample, thus exposing more of the strip surface to the aqueous liquid to be tested. The membrane-biochar strip may additionally include a seal and a string, thread, or grip to permit it to be dropped into a liquid and then removed. The seal may be a string that secures the contents of the membrane-biochar strip at one end by a knot, such as a fiber knot to help shape the bag, or other attachment where the appendage is long enough to permit dipping, swirling, lifting, or other movement of the membrane-biochar strip in an aqueous liquid and for removal of the membrane-biochar strip from a sample holder. A top or distal end of the string, thread or grip, may be attached to a tab which may be colored coded or labelled to permit easy handling and identification of the membrane-biochar strip and the aqueous liquid sample. The string, thread, or grip may be about 4, 5, 6, 7, 8, 9, 10, 11, or 12 cm long, or may be longer or shorter depending on the shape of the sample holder. The string, thread or grip may be made of cotton or another natural fiber, a synthetic fiber such as nylon, or a blend of natural and synthetic fibers which may be woven.

In related embodiments, the membrane-biochar strip may comprise a porous polymeric membrane sheet in the shape of a tube, for example, a hollow fiber membrane, where the ends of the tube are closed in order to contain the aqueous liquid sample. The edges may be closed by an adhesive, a clamp, a tie, or by heat sealing. Alternatively, the porous polymeric membrane sheet may form a balloon shape around the SGBC particles, with the porous polymeric membrane sheet closed at one side, or with the porous polymeric membrane sheet edges tied at one point. Alternatively, the porous polymeric membrane sheet may form a rectangular pillow shape around the SGBC particles. In other embodiments, the edges or perimeter of the porous polymeric membrane sheet may be sealed with an adhesive or folded and mechanically sealed, for example with stitching or stapling. In this embodiment, the four edges may be sealed along each edge, or one edge may be a fold in the porous polymeric membrane sheet with the remaining edges being sealed along each edge.

In some embodiments, each of the two porous polymeric membrane sheets is independently selected from the group including, but are not limited to, poly(vinylidene) fluoride (PVDF), poly(tetrafluoroethylene) (PTFE), poly(acrylonitrile) (PAN), poly(methyl methacrylate) (PMMA), poly(methacrylic acid) (PMAA), poly(acrylic acid) (PAA), poly(vinyl methyl ketone), and poly(ethylene terephthalate) (PET), polysulfone (PSf), poly(ether sulfone) (PSF), polyacrylonitrile (PAN), polyimide (PI), and poly(arylene ether nitrile ketone) (PPENK), which can be used alone or in combination. In some embodiments, each of the two porous polymeric membrane sheets includes one or more polymers selected polypropylene (PP), polyethersulfone (PES), and low-density polyethylene (LDPE). In a preferred embodiment, each of the two porous polymeric membrane sheets includes PP, though other synthetic membranes with similar thickness and porosity may be used. Polypropylene may be a homopolymer or a copolymer, such as a block copolymer or random copolymer. Conveniently, a commercially available polypropylene may be used. In a more preferred embodiment, the polypropylene has a molecular weight in a range of 20,000 to 1,000,000 g/mol, preferably 50,000 to 700,000 g/mol, preferably 100,000 to 500,000 g/mol, or even more preferably about 300,0000 g/mol. Other ranges are also possible. The two porous polymeric membrane sheets may be composed of both polypropylene and polyethylene with a polypropylene to polyethylene weight ratio range of 1:10-10:1, preferably 1:7-7:1, preferably 1:5-5:1, or even more preferably 1:2-2:1. Other ranges are also possible.

In some embodiments, the two porous polymeric membrane sheets of the membrane-biochar strip have an average pore size in a range of 50 μm to 150 μm, including sub-ranges 60 μm, 70 μm, 80 μm, 90 μm, 100 μm, 110 μm, 120 μm, 130 μm, 140 μm, and all sub-ranges in between. Other ranges are also possible. In a preferred embodiment, the two porous polymeric membrane sheets have an average pore size of about 100 μm. In some more preferred embodiments, the membrane-biochar strip includes the two porous polymeric membrane sheets having an average wall thickness of about 100 to 5000 μm, preferably 200-4000 μm, preferably 300-3000 μm, preferably 400-2000 μm, preferably 500-1000 μm, preferably 600-800 μm, or even more preferably 700 μm. Other ranges are also possible.

In one embodiment, the porous polymeric membrane sheets forming the membrane-biochar strip have a uniform thickness of about 1000 μm and a pore size of about 100 μm. In some embodiments, a polypropylene membrane sheet may be replaced by a porous polymeric membrane made of a different kind of plastic or semipermeable membrane. Preferably the porous polymeric membrane has a uniform thickness and pore size that does not vary by more than 0.1, 0.2, 0.5, 1, 2, 3, 4, 5, 6, 7, 8, 9, or 10% over a surface of the porous polymeric membrane exposed to the aqueous liquid and/or organic solvent. Unlike conventional membranes which do not have uniform thickness or porosity, the use of a porous membrane with uniform properties may provide a more accurate and precise adsorption and analyte extraction from the SGBC particles.

In some embodiments, seagrass biochar (SGBC) particles are disposed between two porous polymeric membrane sheets resulting in a sandwich structure. In an embodiment, the two porous polymeric membrane sheets are deposited partially or wholly with at least one layer of the SGBC particles in a uniform and continuous manner. In a preferred embodiment, the SGBC particles form a continuous layer between the two porous polymeric membrane sheets. In an embodiment, the SGBC particles form a monolayer between the two porous polymeric membrane sheets. In another embodiment, the SGBC particles may include more than a single layer between the two porous polymeric membrane sheets. In some embodiments, one or more edges of the sandwich structure is sealed, and the SGBC particles are encapsulated in the membrane-biochar strip. In some embodiments, the SGBC particles have an average particle size of about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, 350 μm, about 360 μm, about 370 μm, about 380 μm, and about 390 μm. Other ranges are also possible. In some further embodiments, the SGBC particles have an average particle size of about 150 μm to 400 μm, more preferably 250 μm. Other ranges are also possible.

In some embodiments, the membrane-biochar strip may further contain a solid sorbent or an adsorbent encapsulated in the sandwich structure formed by the porous polymeric membrane sheets. Examples of solid sorbents include, but are not limited to, silica gel, activated carbon, fly ash, diatomaceous earth, alumina (Al₂O₃), magnesium oxide (MgO), titanium oxide (TiO₂), and polymer sorbents. In a further embodiment, the membrane-biochar strip does not contain a solid sorbent except the SGBC particles (the porous polymeric membrane sheets are not a sorbent for the purposes of the present disclosure). However, in an alternative embodiment, the membrane-biochar strip may incorporate an adsorbent such as a polymeric matrix containing carbon nanotubes. In this alternative embodiment, it is not necessary for the membrane-biochar strip to contain a second layer such as a backing or support layer or a film or applied coating. Such a membrane-biochar strip when configured as an enclosure for the SGBC particles may not contain any loose or packed adsorbent except for the SGBC particles. However, in some alternative embodiments, an adsorbent may be bound to or coated on the porous polymeric membrane sheet.

In some embodiments, about 0.001 to 50, preferably about 0.1 to 40, preferably about 1 to 30, preferably about 5 to 20, or even more preferably about 8 milligrams (mg) of the SGBC particles are present per square centimeters (cm²) exterior surface area of the porous polymeric membrane sheet. Other ranges are also possible. In some embodiments, the membrane-biochar strip may hold a maximum volume of 0.05-10 cm³, preferably 0.1-5 cm³, more preferably 0.25-2.5 cm³ of the SGBC particles. Other ranges are also possible. Preferably, of the exterior surface area of the membrane-biochar strip, at least 80%, preferably at least 90%, more preferably at least 97%, even more preferably about 100% of the is in contact with an aqueous liquid and/or organic solvent during adsorption and desorption, respectively. Other ranges are also possible.

In general, the seagrass may be any suitable seagrass known to one of ordinary skill in the art. The term “seagrass” refers to flowering plants which grow in marine environments in the order Alismatales. There are four families of seagrasses currently recognized: Posidoniaceae, Zosteraceae, Hydrocharitaceae and Cymodoceaceae. A seagrass from any of these families may be used. In preferred embodiments, the seagrass is a member of the family Cymodoceaceae. This family includes 17 species of seagrass, grouped into five genera. These genera are Syringodium, Cymodocea, Amphibolis, Thalassodendron, and Halodule. A seagrass from any of these genera may be used. In preferred embodiments, the seagrass is a member of the genus Halodule. The genus Halodule is common in tropical and semi-tropical oceans and can be found in shores of all continents except Europe and Antarctica. The genus is comprised of six species: Halodule bermudensis, (found primarily in Bermuda), Halodule ciliate (found primarily in Panama), Halodule emarginata (found primarily in Brazil), Halodule pinifolia (found primarily in the Indian and Pacific Oceans off the costs of, for example, India, Sri Lanka, Southeast Asia, Hainan, Taiwan, Ryukyu Islands, New Guinea, Queensland, Fiji, New Caledonia, Tonga, and the Caroline Islands), Halodule uninervis (also found primarily in the Indian and Pacific Oceans, as well as the Red Sea, Persian Gulf, Bay of Bengal), and Halodule wrightii (found primarily in the Atlantic Ocean, especially the Caribbean and Gulf of Mexico). In some other embodiments, the seagrass biochar (SGBC) particles may be prepared from Zostera Marina seagrass, Posidonia Oceanica seagrass, Halodule Uninervis seagrass, Thalassia Testudinum seagrass, Syringodium Filiforme seagrass, Enhalus Acoroides seagrass, Cymodocea Nodosa seagrass, Thalassodendron Ciliatum seagrass, Phyllospadix Spp seagrass, and Posidonia Australis seagrass. In some embodiments, the raw seagrass is Halodule Uninervis seagrass. In the present disclosure, the SGBC particles are prepared from Halodule Uninervis seagrass.

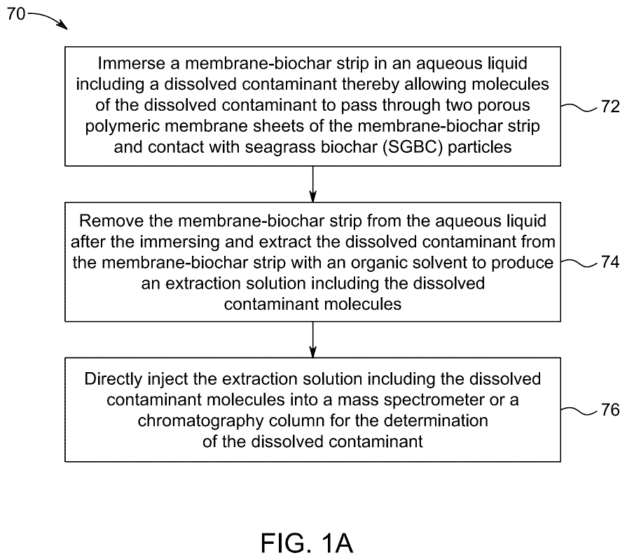

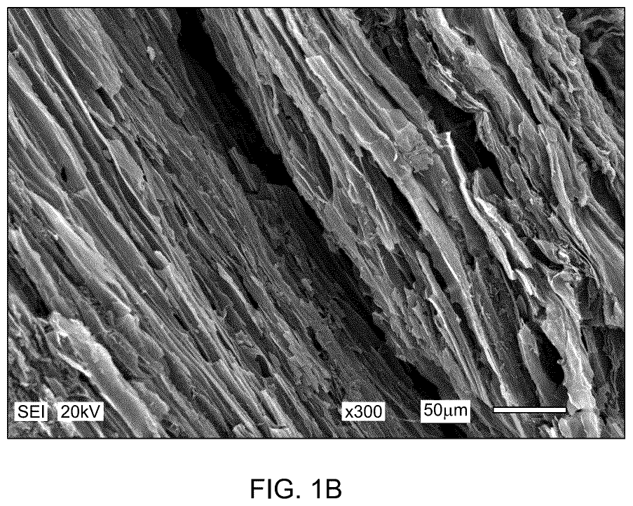

In some embodiments, the SGBC particles have a porous structure, including a plurality of fractures and grooves, as depicted in FIG. 1B. In some embodiments, the SGBC particles have a long dimension of 100 to 1000 μm, preferably 150 to 800 μm, preferably 200 to 600 μm, or even more preferably about 250 μm. Other ranges are also possible. In some embodiments, the SGBC particles have a thickness of about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, and about 4.9 μm. Other ranges are also possible. In some embodiments, the SGBC particles have a width of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, and about 24 μm. Other ranges are also possible. In some preferred embodiments, the SGBC particles have a long dimension of 250 μm, a thickness of about 0.5 μm to 5 μm, and a width of 5 to 25 μm. Other ranges are also possible.

A shown in FIG. 1B, the SGBC particles may represent agglomerations of sheet-like forms. Individual sheets may have a thickness of 100 nm to 5 μm, preferably 500 nm to 2 μm or about 1 μm. The sheet-like forms have a width of 5-20 μm, preferably 10-15 μm. The sheet-like forms are densely spaced such that 5-50, preferably 10-25 or about 10 sheets are present in a 50 μm section measured perpendicular to the axis of the sheets. The spacing between the sheets thus represents voids, fractures or grooves.

As used herein, the term “adsorption/desorption method” generally refers to a technique used to measure the specific surface area of a solid material, such as the SGBC particles. In some embodiments, the SGBC particles are exposed to a stream of gas, e.g., nitrogen (N₂), at low temperature and pressure. The gas is adsorbed onto the surface of the SGBC particles, filling the pores and creating a monolayer of adsorbed gas molecules. In some further embodiments, the amount of gas molecules adsorbed at a given pressure is measured using a gas adsorption instrument, such as an AutosorbiQ Quantachrome instrument. In some preferred embodiments, the BET analysis is performed on an analyzer according to the software manual. In some more preferred embodiments, the gas is gradually removed from the SGBC particles, causing the desorption of the adsorbed gas molecules. The amount of gas molecules desorbed at a given pressure is also measured using the gas adsorption instrument. By analyzing the amount of gas adsorbed and desorbed, the specific surface area of the SGBC particles can be calculated using the BET (Brunauer-Emmett-Teller) and Barrett, Joyner and Halenda (BJH) equation.

In some embodiments, the SGBC particles have a surface area of about 50 square meters per gram (m²/g), about 51 m²/g, about 52 m²/g, about 53 m²/g, about 54 m²/g, about 55 m²/g, about 56 m²/g, about 57 m²/g, about 58 m²/g, about 59 m²/g, about 60 m²/g, about 61 m²/g, about 62 m²/g, about 63 m²/g, about 64 m²/g, about 65 m²/g, about 66 m²/g, about 67 m²/g, about 68 m²/g, and about 69 m²/g. Other ranges are also possible. In some embodiments, the SGBC particles have a surface area of about 50 m²/g to 70 m²/g. In some embodiments, the SGBC particles have a pore volume of about 0.05 cubic centimeters per gram (cm³/g), about 0.06 cm³/g, about 0.07 cm³/g, about 0.08 cm³/g, and about 0.09 cm³/g. In some embodiments, the SGBC particles have a pore volume of about 0.05 to 0.1 cm³/g, and all sub-ranges in between.

In some embodiments, the SGBC particles have an absorption capacity of up to 100, preferably up to 80, preferably up to 60, preferably up to 40, or even more preferably up to 20 milligrams (mg) of a phenolic compound per gram of the SGBC particles. Other ranges are also possible.

In some embodiments, the SGBC particles includes about 58 wt. %, about 59 wt. %, about 60 wt. %, about 61 wt. %, about 62 wt. %, about 63 wt. %, about 64 wt. %, about 65 wt. %, about 66 wt. %, and about 67 wt. % C; about 10 wt. %, about 11 wt. %, about 12 wt. %, about 13 wt. %, and about 14 wt. % O; about 5 wt. %, about 6 wt. %, about 7 wt. %, about 8 wt. %, about 9 wt. %, about 10 wt. %, about 11 wt. %, about 12 wt. %, about 13 wt. %, and about 14 wt. % K; about 2 wt. %, about 3 wt. %, about 4 wt. %, about 5 wt. %, about 6 wt. %, about 7 wt. %, about 8 wt. %, about 9 wt. %, about 10 wt. %, and about 11 wt. % Cl; about 1 wt. %, about 2 wt. %, about 3 wt. %, and about 4 wt. % Ca; about 1 wt. %, about 2 wt. %, about 3 wt. %, and about 4 wt. % S; about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, and about 1.9 wt. % Mg, each wt. % based on the total weight of the SGBC particles, as determined by energy dispersive X-ray (EDX) analysis. In some embodiments, the SGBC particles include 58 to 68 wt. % C, more preferably 63.7; 10 to 15 wt. % O, more preferably 12.3 wt. %; 5 to 15 wt. % K; more preferably 10.6 wt. %; 2 to 12 wt. % Cl, more preferably 7.8 wt. %; 1 to 5 wt. % Ca, more preferably 2.4 wt. %; 1 to 5 wt. % S, more preferably 2.2 wt. %; 0.1 to 2 wt. % Mg, more preferably 1.0 wt. %; each wt. % based on the total weight of the SGBC particles, as determined by EDX analysis.

FIG. 1A illustrates a flow chart of a method 70 for the determination of a dissolved contaminant in an aqueous liquid. The order in which the method 70 is described is not intended to be construed as a limitation, and any number of the described method steps can be combined in any order to implement the method 70. Additionally, individual steps may be removed or skipped from the method 70 without departing from the spirit and scope of the present disclosure.

At step 72, the method 70 includes immersing the membrane-biochar strip in the aqueous liquid, including the dissolved contaminant, thereby allowing molecules of a dissolved contaminant to pass through the two porous polymeric membrane sheets of the membrane-biochar strip and contact with the SGBC particles. In some embodiments, the aqueous liquid may be tap water, distilled water, bidistilled water, deionized water, deionized distilled water, reverse osmosis water, and/or some other water.

In some embodiments, the dissolved contaminant may be a phenolic compound, a dye, a polycyclic aromatic hydrocarbon, an herbicide, a pesticide, a persistent organic pollutant, or the like.

In some embodiments, the dissolved contaminant is a phenolic compound. A phenolic compound is an organic compound having a hydroxyl group (—OH) bonded directly to an aromatic hydrocarbon group. Examples of phenolic compounds include, but are not limited to, phenol (the namesake of the group of compounds), bisphenols (including bisphenol A), butylated hydroxytoluene (BHT), 4-nonylphenol, orthophenyl phenol, picric acid, phenolphthalein and its derivatives mentioned above, dimethylphenols (also called xylenols, including 2,3-dimethylphenol, 2,4-dimethylphenol, 2,5-dimethylphenol, 2,6-dimethylphenol, 3,4-dimethylphenol, and 3,5-dimethylphenol), diethylstilbestrol, L-DOPA, propofol, butylated hydroxyanisole, 4-tert-butylcatechol, tert-butylhydroquinone, carvacrol, chloroxyleol, cresol (including M-, O-, and P-cresol), 2,6-di-tert-butylphenol, 2,4-dimethyl-6-tert-butylphenol, 2-ethyl-4,5-dimethylphenol, 4-ethylguaiacol, 3-ethylphenol, 4-ethylphenol, flexirubin, mesitol, 1-nonyl-4-phenol, thymol, 2,4,6-tri-tert-butylphenol, chlorophenol (including 2-, 3-, and 4-chlorophenol), dichlorophenol (including 2,4- and 2,6-dichlorophenol), bromophenol, dibromophenol (including 2,4-dibromophenol), nitrophenol, norstictic acid, oxybenzone, and paracetamol (also known as acetoaminophen). In some preferred embodiments, the dissolved contaminant is at least one phenolic compound selected from phenol, 2-chlorophenol (2-CP), 2-methyl phenol (2-MP), 2,4-dimethyl phenol (2,4-DMP), 2,4-dichlorophenol (2,4-DCP), and 2-nitro phenol (2-NP). In some embodiments, the phenolic compound may include vanillin, ferulic acid, gallic acid, phenolic acid, catechin, lignan, ferulic acid, quercetin, resveratrol, benzoic acid, caffeic acid, carvacrol, luteolin, kaempferol, flavonols, and p-coumaric acid.

In some embodiments, the dissolved contaminant is a dye. A dye is a colored substance that chemically binds to a material it may be intended to color. Generally, a dye is applied in solution, typically aqueous solution. Examples of dyes include, but are not limited to: acridine dyes, which are acridine and its derivatives such as acridine orange, acridine yellow, acriflavine, and gelgreen; anthraquinone dyes, which are anthraquinone and its derivatives such as acid blue 25, alizarin, anthrapurpurin, carminic acid, 1,4-diamno-2,3-dihydroanthraquinone, 7,14-dibenzypyrenequinone, dibromoanthrone, 1,3-dihydroxyanthraquinone, 1,4-dihydroxyanthraquinone, disperse red 9, disperse red 11, indanthrone blue, morindone, oil blue 35, parietin, quinizarine green SS, remazol brilliant blue R, solvent violet 13, 1,2,4-trihydroxyanthraquinone, vat orange 1, and vat yellow 1; diaryl methane dyes such as auramine O, triarylmethane dyes such as acid fuchsin, aluminon, aniline blue WS, aurin, aurintricarboxylic acid, brilliant blue FCF, brilliant green, bromocresol green, bromocresol purple, bromocresol blue, bromophenol blue, bromopyrogallol red, chlorophenol red, coomassie brilliant blue, cresol red, O-cresolphthalein, crystal violet, dichlorofluorescein, ethyl green, fast green FCT, FIASH-EDT2, fluoran, fuchsine, green S, light green SF, malachite green, merbromin, metacresol purple, methyl blue, methyl violet, naphtholphthalein, new fuchsine, pararosaniline, patent blue V, phenol red, phenolphthalein, phthalein dye, pittacal, spirit blue, thymol blue, thymolphthalein, Victoria blue BO, Victoria blue R, water blue, xylene cyanol, and xylenol orange; azo dyes such as acid orange 5, acid red 13, alican yellow, alizarine yellow R, allura red AC, amaranth, amido black 10B, aniline yellow, arylide yellow, azo violet, azorubine, basic red 18, biebrich scarlet, Bismarck brown Y, black 7984, brilliant black BN, brown FK, chrysoine resorcinol, citrus red 2, congo red, D&C red 33, direct blue 1, disperse orange 1, eriochrome black T, evans blue, fast yellow AB, orange 1, hydroxynaphthol blue, janus green B, lithol rubine BK, metanil yellow, methyl orange, methyl red, methyl yellow, mordant brown 33, mordant red 19, naphthol AS, oil red O, oil yellow DE, orange B, orange G, orange GGN, para red, pigment yellow 10, ponceau 2R, prontosil, red 2G, scarlet GN, Sirius red, solvent red 26, solvent yellow 124, sudan black B, sudan I, sudan red 7B, sudan stain, tartrazine, tropaeolin, trypan blue, and yellow 2G; phthalocyanine dyes such as phthalocyanine blue BN, phthalocyanine Green G, Alcian blue, and naphthalocyanine, azin dyes such as basic black 2, mauveine, neutral red, Perkin's mauve, phenazine, and safranin; indophenol dyes such as indophenol and dichlorophenolindophenol; oxazin dyes; oxazone dyes; thiazine dyes such as azure A, methylene blue, methylene green, new methylene blue, and toluidine blue; thiazole dyes such as primuline, stains-all, and thioflavin; xanthene dyes such as 6-carboxyfluorescein, eosin B, eosin Y, erythosine, fluorescein, rhodamine B, rose bengal, and Texas red; fluorone dyes such as calcein, carboxyfluorescein diacetate succinimidyl ester, fluo-3, fluo-4, indian yellow, merbromin, pacific blue, phloxine, and seminaphtharhodafluor; or rhodamine dyes such as rhodamine, rhodamine 6G, rhodamine 123, rhodamine B, sulforhodamine 101, and sulforhodamine B.

In some embodiments, the dissolved contaminant is a polycyclic aromatic hydrocarbon. A polycyclic aromatic hydrocarbon (PAH) is an aromatic hydrocarbon composed of multiple aromatic rings. Examples of polycyclic aromatic hydrocarbons include naphthalene, anthracene, phenanthrene, phenalene, tetracene, chrysene, triphenylene, pyrene, pentacene, benzo[a] pyrene, corannulene, benzo[g,h,i] perylene, coronene, ovalene, benzo[c] fluorine, acenaphthene, acenaphthylene, benz[a] anthracene, benzo[b] fluoranthene, benzo[j] fluoranthene, benzo[k] fluoranthene, benzo[e] pyrene, cyclopenta[c,d] pyrene, dibenz[a,h] anthracene, dibenzo[a,e] pyrene, dibenzo[a,h] pyrene, dibenzo[a,i] pyrene, dibenzo[a,1] pyrene, fluoranthene, fluorine, indeno[1,2,3-c,d] pyrene, 5-methylchrysene, naphthacene, pentaphene, picene, and biphenylene.

In some embodiments, the dissolved contaminant is an herbicide. An herbicide (also known as “weedkiller”) is a substance that is toxic to plants and may kill, inhibit the growth of, or prevent the germination of plants. Herbicides are typically used to control the growth of or remove unwanted plants from an area of land, particularly in an agricultural context. Examples of herbicides include, but are not limited to, 2,4-D, aminopyralid, chlorsulfuron, clopyralid, dicamba, diuron, glyphosate, hexazinone, imazapic, imazapyr, methsulfuron methyl, picloram, sulfometuron methyl, triclopyr, fenoxaprop, fluazifop, quizalofop, clethodim, sethoxydim, chlorimuron, foramsulfuron, halosulfuron, nicosulfuron, primisulfuron, prosulfuron, rimsulfuron, thofensulfuron, tribenuron, imazamox, imazaquin, flumetsulam, cloransulam, thiencarbazone, fluoxpyr, diflufenzopyr, atrazine, simazine, metribuzin, bromoxynil, bentazon, linuron, glufosinate, clomazone, isoxaflutole, topramezone, mesotrione, tembotrione, acifluorfen, formesafen, lactofen, flumiclorac, flumioxazin, fulfentrazone, carfentrazone, fluthiacet-ethyl, falufenacil, paraquat, ethalfluralin, pendimethalin, trifluralin, butylate, EPTC, ecetochlor, alachlor, metolachlor, dimethenamid, flufenacet, and pyroxasulfone.

In some embodiments, the dissolved contaminant is a pesticide. A pesticide is a substance meant to prevent, destroy, or control pests including, but not limited to algae, bacteria, fungi, plants, insects, mites, snails, rodents, and viruses. A pesticide intended for use against algae is known as an algicide. Examples of algicides include benzalkonium chloride, bethoxazin, cybutryne, dichlone, dichlorophen, diuron, endothal, fentin, isoproturon, methabenthiazuron, nabam, oxyfluorfen, pentachlorophenyl laurate, quinoclamine, quinonamid, simazine, terbutryn, and tiodonium.

A pesticide intended for use against bacteria is known as a bactericide. Examples of bactericides include antibiotics such as: aminoglycosides such as amikacin, gentamicin, kanamycin, neomycin, netilmicin, tobramycin, paromomycin, streptomycin, and spectinomycin; ansamycins such as geldanamycin, herbimycin, and rifaximin; carbacephems such as loracarbef; carbapenems such as ertapenem, doripenem, imipenem, and meropenem; cephalosporins such as cefadroxil, cefazolin, cephradine, cephapirin, cephalothin, cephalexin, cefaclor, cefoxitin, cefotetan, cefamandole, cefmetazole, cefonicid, cefprozil, cefuroxime, cefixime, cefdinir, cefditoren, cefoperazone, cefotaxime, cefpodoxime, cefazidime, ceftibuten, ceftizoxime, moxalactam, ceftriaxone, cefepime, cefaroline fosamil, and ceftobiprole; glycopeptides such as teicoplanin, vancomycin, telavancin, dalbavancin, and oritavancin; lincosamides such as clindamycin and lincomycin; lipopeptides such as daptomycin; macrolides such as azithromycin, clarithromycin, erythromycin, roxithromycin, telithromycin, spiramycin, and fidoxamicin; monobactams such as aztreonam; nitrofurans such as furazolidone and nitrofurantoin; oxazolidinones such as linezolid, posizolid, radezolid, and torezolid; penicillins such as amoxicillin, ampicillin, azlocillin, dicloxacillin, flucloxacillin, mezlocillin, methicillin, nafcillin, oxacillin, penicillins (including penicillin G and V), piperacillin, temocillin, and ticarcillin; polypeptides such as bacitracin, colistin, and polymyxin B; quinolones such as ciproflaxacin, enoxacin, gatifloxacin, gemifloxacin, levofloxacin, lomefloxacin, moxifloxacin, nadifloxacin, nalidixic acid, norfloxacin, ofloxacin, trovafloxacin, gepafloxacin, sparfloxacin, and temafloxacin; sulfonamides such as mafenide, sulfacetamide, sulfadiazine, sulfadithoxine, sulfamethizole, sulfamethoxazole, sulfanilamide, sulfasalazine, sulfisoxazole, and sulfonamidochrysoidine; tetracyclines such as demeclocycline, doxycycline, metacycline, minocycline, oxytetracycline, and tetracycline.

A pesticide intended for use against fungi is known as a fungicide. Examples of fungicides include acibenzolar, acypetacs, aldimorph, anilazine, aureofungin, azaconazole, azithiram, azoxystrobin, benalaxyl, benodanil, benomyl, benquinox, benthiavalicarb, binapacryl, biphenyl, bitertanol, bixafen, blasticidin-S, boscalid, bromuconazole, captafol, captan, carbendazim, carboxin, carpropamid, chloroneb, chlorothalonil, chlozolinate, cyazofamid, cymoxanil, cyprodinil, dichlofluanid, diclocymet, dicloran, diethofencarb, difenoconazole, diflumetorim, dimethachlone, dimethomorph, diniconazole, dinocap, dodemorph, edifenphos, enoxastrobin, epoxiconazole, etaconazole, ethaboxam, ethirimol, etridiazole, famoxadone, fenamidone, fenarimol, fenbuconazole, fenfuram, fenhexamid, fenoxanil, fenpropidin, fenpropimorph, ferbam, fluazinam, fludioxonil, flumorph, fluopicolide, fluopyram, fluoroimide, fluoxastrobin, flusilazole, flutianil, flutolain, flopet, fthalide, furalaxyl, guazatine, hexaconazole, hymexazole, imazalil, imibenconazole, iminoctadine, iodocarb, ipconazole, iprobenfos, iprodione, iprovalicarb, siofetamid, isoprothiolane, isotianil, kasugamycin, laminarin, mancozeb, mandestrobin, mandipropamid, maneb, mepanypyrim, mepronil, meptyldinocap, mealaxyl, metominostrobin, metconazole, methafulfocarb, metiram, metrafenone, myclobutanil, naftifine, nuarimol, octhilinone, ofurace, orysastrobin, oxadixyl, oxathiapiprolin, oxolinic acid, oxpoconazole, oxycarboxin, oxytetracycline, pefurazate, penconazole, pencycuron, penflufen, penthiopyrad, phenamacril, picarbutrazox, picoxystrobin, piperalin, polyoxin, probenzole, prochloraz, procymidone, propamocarb, propiconazole, propineb, proquinazid, prothiocarb, prothioconazole, pydiflumetofen, pyraclostrobin, pyrametostrobin, pyraoxystrobin, pyrazophos, pyribencarb, pyributicarb, pyrifenox, pyrimethanil, pyrimorph, pyriofenone, pyroquilon, quinoxyfen, quintozene, sedaxane, silthiofam, simeconazole, spiroxamine, streptomycin, tebuconazole, tebufloquin, teclofthalam, tecnazene, terbinafine, tetraconazole, thiabendazole, thifluzamide, thiphanate, thiram, tiadinil, tolclosfos-methyl, folfenpyrid, tolprocarb, tolylfluanid, triadimefon, triadimenol, triazoxide, triclopyricarb, tricyclazole, tridemorph, trifloxystrobin, triflumizole, triforine, validamycin, and vinclozolin.

A pesticide intended for use against plants is known as an herbicide as described above.

A pesticide intended for use against insects is known as an insecticide. Examples of insecticides are: organochlorides such as Aldrin, chlordane, chlordecone, DDT, dieldrin, endofulfan, endrin, heptachlor, hexachlorobenzene, lindane, methoxychlor, mirex, pentachlorophenol, and TDE; organophosphates such as acephate, azinphos-methyl, bensulide, chlorethoxyfos, chlorpyrifos, diazinon, chlorvos, dicrotophos, dimethoate, disulfoton, ethoprop, fenamiphos, fenitrothion, fenthion, malathion, methamdophos, methidathion, mevinphos, monocrotophos, naled, omethoate, oxydemeton-methyl, parathion, phorate, phosalone, phosmet, phostebupirim, phoxim, pirimiphos-methyl, profenofos, terbufos, and trichlorfon; carbamates such as aldicarb, bendiocarb, carbofuran, carbaryl, dioxacarb, fenobucarb, fenoxycarb, isoprocarb, methomyl; pyrethroids such as allethrin, bifenthrin, cyhalothrin, cypermethrin, cyfluthrin, deltamethrin, etofenprox, fenvalerate, permethrin, phenothrin, prallethrin, resmethrin, tetramethrin, tralomethrin, and transfluthrin; neonicotinoids such as acetamiprid, clothiandin, imidacloprid, nithiazine, thiacloprid, and thiamethoxam; ryanoids such as chlorantraniliprole, cyanthaniliprole, and flubendiamide.

A pesticide intended for use against mites is known as a miticide. Examples of miticides are permethrin, ivermectin, carbamate insecticides as described above, organophosphate insecticides as described above, dicofol, abamectin, chlorfenapyr, cypermethrin, etoxazole, hexythiazox, imidacloprid, propargite, and spirotetramat.

A pesticide intended for use against snails and other mollusks is known as a molluscicide. Examples of molluscicides are metaldehyde and methiocarb.

A pesticide intended for use against rodents is known as a rodenticide. Examples of rodenticides are warfarin, coumatetralyl, difenacoum, brodifacoum, flocoumafen, bromadiolone, diphacinone, chlorophacinone, pindone, difethialone, cholecalciferol, ergocalciferol, ANTU, chloralose, crimidine, 1,3-difluoro-2-propanol, endrin, fluroacetamide, phosacetim, pyrinuron, scilliroside, strychnine, tetramethylenedisulfotetramine, bromethalin, 2,4-dinitrophenol, and uragan D2.

A pesticide intended for use against viruses is known as a virucide. Examples of virucides are cyanovirin-N, griffithsin, interferon, NVC-422, scytovirin, urumin, virkon, zonroz, and V-bind viricie.

In some embodiments, the dissolved contaminant is a persistent organic pollutant. A persistent organic pollutant is a toxic organic chemical that adversely affects human and environmental health, can be transported by wind and water, and can persist for years, decades, or centuries owing to resistance to environmental degradation by natural chemical, biological, or photolytic processes. Persistent organic pollutants are regulated by the United Nations Environment Programme 2001 Stockholm Convention on Persistent Organic Pollutants. Examples of persistent organic pollutants are Aldrin, chlordane, dieldrin, endrin, heptachlor, hexachlorobenzene, mirex, toxaphene, polychlorinated biphenyl (PCBs), dichlorodiphenyltrichloroethane (DDT), dioxins, polychlorinated dibenzofurans, chlordecone, hexachlorocyclohexane (α- and β-), hexabromodiphenyl ether, lindane, pentachlorobenzene, tetrabromodiphenyl ether, perfluorooctanesulfonic acid, endosulfans, and hexabromocyclododecane.

In some embodiments, the concentration of the dissolved contaminant in the aqueous liquid is in a range of 1 milligram per liter (mg/L), about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L of the aqueous liquid. Other ranges are also possible. In some embodiments, the dissolved contaminant is present in the aqueous liquid at a concentration of about 1 mg/L to 20 mg/L of the aqueous liquid. Other ranges are also possible.

In some embodiments, the method 70 further includes preparing the SGBC particles by washing raw seagrass and heating at a temperature of about 500 degrees Celsius (C) in an atmosphere of an inert gas to form the SGBC in the form of particles. In some embodiments, the seagrass may be used in the method substantially as collected (e.g., from a natural source or cultivated source). In some embodiments, the raw seagrass is Halodule Uninervis seagrass. In some embodiments, the washing of the raw seagrass can be done with ethanol or water. In some other embodiments, the washing may also be performed with an aqueous surfactant solution. The surfactant may be any suitable surfactant known to one of ordinary skill in the art. In some embodiments, the seagrass may be dried prior to the heating. Such drying may be performed at any suitable temperature. For example, the drying may be freeze drying, which takes place at or below the freezing point of water (i.e., 0° C.). In another example, the drying may take place at ambient temperature (e.g., from about 20 to about 25° C.). In another example, the drying may take place at elevated temperature (e.g., from about 50° C. to about 100° C.). Preferably, the drying reduces a moisture content of the seagrass to below 10%, preferably below 9.5%, preferably below 9%, preferably below 8.5%, preferably below 8%, preferably below 7.5%, preferably below 7%, preferably below 6.5%. Other ranges are also possible.

In some embodiments, the seagrass may be reduced to small particles prior to the heating. In general, the reducing to small particles may be performed by any suitable technique or with any suitable equipment known to one of ordinary skill in the art. Examples of such techniques include, but are not limited to, grinding, ball milling, chopping, pulverizing, crushing, pounding, mincing, shredding, smashing, and fragmenting. In some embodiments, the reducing to small particles may take place using a mill, ball mill, rod mill, autogenous mill, cutting mill, semi-autogenous grinding mill, pebble mill, buhrstone mill, burr mill, tower mill, vertical shaft impactor mill, a low energy milling machine, grinder, pulverizer, mortar and pestle, blender, crusher, or other implement used to reduce a material to small particles.

In some embodiments, the seagrass is not reduced to small particles prior to use in the method.

In some embodiments, the heating can be performed by using heating appliances such as ovens, microwaves, autoclaves, hot plates, heating mantles and tapes, oil baths, salt baths, sand baths, air baths, hot-tube furnaces, and hot-air guns. The inert gas may include, but are not limited to, argon (Ar), helium (He), and nitrogen (N₂), and they may be used in the following combinations: Ar/He, Ar/He/N₂, and N₂/He. In some embodiments, the raw seagrass includes Zostera Marina seagrass, Posidonia Oceanica seagrass, Halodule Uninervis seagrass, Thalassia Testudinum seagrass, Syringodium Filiforme seagrass, Enhalus Acoroides seagrass, Cymodocea Nodosa seagrass, Thalassodendron Ciliatum seagrass, Phyllospadix Spp seagrass, and Posidonia Australis seagrass.

In some embodiments, the inert gas is introduced at a flow rate of about 10 to 100 mL/min, preferably 20 to 80 mL/min, preferably 30 to 60 mL/min, or even more preferably about 40 mL/min. Other ranges are also possible.

At step 74, the method 70 includes removing the membrane-biochar strip from the aqueous liquid after immersing and extracting the dissolved contaminant from the membrane-biochar strip with an organic solvent to produce an extraction solution including the dissolved contaminant molecules. In some embodiments, the organic solvent may include, but is not limited to, tetrahydrofuran, ethyl acetate, dimethylformamide, acetonitrile, acetone, dimethyl sulfoxide, nitromethane, propylene carbonate, ethanol, formic acid, n-butanol, methanol, or any combination thereof. In some embodiments, the organic solvent is at least one selected from the group consisting of benzene, cyclohexane, ethanol, methanol, acetone, ethyl acetate, dichloromethane, toluene, and diethyl ether.

In a preferred embodiment, the extracting may be performed by sonicating a vial containing the organic solvent, the membrane-biochar strip containing the dissolved contaminant, using a sonicator. In this embodiment, the extraction time may be considered the duration of the sonication. Here, the vial is sonicated for a period of 5-60 min, preferably 10-50 min, more preferably 15-40 min, 20-30 min, 21-29 min, 22-27 min, even more preferably about 25 min. The sonicating may be continuous, pulsed, or modulated in some way. Preferably the sonicating is continuous. The sonicator used may be a probe sonicator inserted into the mixture, or, more preferably, a bath sonicator that can sonicate a plurality of samples without a direct contact between the sonicator and the samples. Without such a direct contact, the chance of cross-contamination is reduced, and cleaning of the sonicator between different samples is not needed. The sonicating frequency is about 20-120 kHz, about 40-100 kHz, or about 60-90 kHz, and may be considered “ultrasonication.” However, in an alternative embodiment, a sonicator may create vibrations at a lower frequency than ultrasonic, for example, sonic vibrations, which may be used for the same purpose. The sonicating power may be 20-100 W, preferably 40-80 W, 45-75 W, more preferably 50-70 W, even more preferably 55-65 W, or about 60 W. Other ranges are also possible.

In one embodiment of the method, the extraction of the dissolved contaminant occurs at the same time as shaking, and/or sonicating, which means that the entire extraction method occurs in a single operation. For example, the dissolved contaminant may be extracted from the SGBC particles inside the membrane-biochar strip and into the organic solvent passing thorough the porous polymeric membrane sheets, without requiring a researcher to perform those extraction steps separately or subsequently. Additionally, in this embodiment, parts of the dissolved contaminant may be simultaneously partitioned to two locations within the vial: in the SGBC particles inside the membrane-biochar strip, and within the organic solvent outside the membrane-biochar strip but within the vial. In addition, the dissolved contaminant may be present in the headspace of the vial as a vapor. In another embodiment, the dissolved contaminant may be limited to different locations in the vial throughout the method. For example, at the beginning of the extracting, the dissolved contaminant may exist only in the organic solvent and within the membrane-biochar strip. With prolonged extracting, e.g., shaking, analyte may exist in all four places as previously described. Likewise, an internal standard may simultaneously exist all locations within the vial, or may be limited to one or two locations. In one embodiment, an internal standard may be mixed with the organic solvent.

The amount of the dissolved contaminant or internal standard in the phases of the strip, solution, vapor, and liquid-phase extraction medium may depend on initial concentrations, the amount and rate of heating and agitating, and the partition coefficient of the dissolved contaminant or internal standard among the different phases. Where two or more dissolved contaminants are present, their relative concentrations may vary across different phases. Likewise, where one or more internal standards are present, the relative concentrations between an internal standard and a dissolved contaminant, or between two internal standards, may also vary across different phases. In some embodiments, the extracting may be performed under heating at a temperature of 40-80° C., preferably 45-70° C., for 1-4 hours, or by placing in a desiccator or vacuum desiccator. Other ranges are also possible.

In one embodiment, during the method, a temperature of the organic solvent does not exceed 50° C., preferably does not exceed 48° C., more preferably does not exceed 45° C. In one embodiment, the sample may be cooled during the shaking, and/or sonicating, for instance, by controlling the temperature of the water bath in a bath sonicator. In other embodiments, a maximum temperature reached by the organic solvent may be in a range of 30-80° C., preferably 35-75° C., more preferably 40-70° C., or less than 65° C. In a related embodiment, a maximum temperature reached by the organic solvent may be no greater than 70° C. Other ranges are also possible.

In some embodiments, after the removing the membrane-biochar strip from the organic solvent to produce an extraction solution containing the dissolved contaminant. The extraction solution may be further dried to produce a concentrated extract solution. In some embodiments, the drying may be performed with a rotary evaporator, or by letting the vial sit open on a lab bench or in a fume hood. In one embodiment, the concentrated extract solution is located in a bottom portion of the vial. In some embodiments, the drying is carried out at 20 to 100° C., preferably 30 to 80° C., or even more preferably 40 to 70° C., to produce the concentrated extract solution. Other ranges are also possible.

In another embodiment, the concentrated extract solution may be further dried to form a dried extract. The dried extract may then be resuspended in a second volume of a second organic solvent to produce a second concentrated extract solution, for instance, when the second volume of the second organic solvent has a volume is less than the organic solvent used for the extraction. In one embodiment, the second volume may have a volume that is 0.5-60%, preferably 1.0-30%, more preferably 2.0-20%, even more preferably about 10% of the volume of the organic solvent used for the extraction. In some embodiments, a different organic solvent may be used for the concentrated extract solution as compared to the organic solvent used for the extraction. In alternative embodiments, the extract may be diluted to a greater volume (and thus lower concentration of extract) than the original volume of the organic solvent used for the extraction. Preferably, no filtering step and/or centrifuging step is used on the organic solvent, extract, or concentrated extract solution.

At step 76, the method 70 includes directly injecting the extraction solution including the dissolved contaminant molecules into a mass spectrometer or a chromatography column for the determination of the dissolved contaminant. In some embodiments, the method has a sampling rate (Rₛ) in a range of 0.005 to 0.045 liter per day (L/day) per 100 milligrams (mg) of the SGBC particles encapsulated in the membrane-biochar strip.

In one embodiment, the mass spectrometer or chromatography column may be part of a GCMS. In one embodiment, a portion of the extraction solution may be injected directly into a GCMS without forming a concentrated extract solution. In one embodiment, a typical commercial GCMS may be used. The gas chromatography may be coupled with a single mass spectrometer (i.e., GC-MS) or with a plurality of mass spectrometers, i.e., tandem mass spectrometry, such as GC-MS-MS. During the mass spectrometry, the dissolved contaminant molecules may be fragmented by either positive chemical ionization, for example, with methanol as the chemical ionization reagent, or preferably electron ionization (such as GC-EI-MS). In other embodiments, the detection and quantification of the concentration of analytes in the solid sample is via gas chromatography coupled with a flame ionization detector (FID), a thermal energy detector, a nitrogen-phosphorus detector, or a nitrogen chemiluminescence detector. The carrier gas may be nitrogen, helium, and/or hydrogen. Preferably the carrier gas is helium with a purity of greater than 99.9 mol %, preferably greater than 99.99 mol %, more preferably greater than 99.999 mol %.

The stationary phase of the gas chromatography column may be comprised of a methyl siloxane (also known as methyl polysiloxane or dimethyl polysiloxane), phenyl polysiloxane, dimethyl arylene siloxane, cyanopropylmethyl polysiloxane, and/or trifluoropropylmethyl polysiloxane with a film thickness of 0.10-7 μm, preferably 0.15-1 μm, more preferably 0.2-0.5 μm. The column length may be 10-120 m, preferably 15-50 m, more preferably 25-40 m, with an inside diameter of 0.08-0.60 mm, preferably 0.15-0.40 mm, more preferably 0.20-0.30 mm.

In some embodiments, a helium gas flow of purity about 99.999% was used as carrier gas with a flow rate of, e.g., preferably about 1.2 mL/min and the total run time was, e.g., preferably about 27 min. The extract of a volume, e.g., preferably about 1.0 μL was injected using split-less mode. In some embodiments, the injector temperature may be maintained at, e.g., preferably about 300° C. In some embodiments, the column oven temperature was ramped from, e.g., preferably about 70° C. after a holding time of, e.g., preferably about 1 min to, e.g., preferably about 200° C. at a rate of, e.g., preferably about 10° C./min. Then another ramping was set from, e.g., preferably 200° C. to 250° C. at a rate of, e.g., preferably about 5° C./min with a holding time of, e.g., preferably 3 min. The temperature of the detector was set at, e.g., preferably about 250° C. Other ranges are also possible.

The parameters of a GCMS instrument and method of operation, including but not limited to flow rate, temperature, temperature gradient, run time, pressure, sample injection, sample volume, ionization method, ionization energy, and scanning range may be adjusted by a person of ordinary skill in the art to account for differences in samples, equipment, and techniques.

The dissolved contaminant molecules may be detected by monitoring a known elution time and/or m/z (mass to charge ratio) for a positive signal as compared with a blank sample. An internal standard's m/z may depend on its identity. For quantitation, known concentrations of an internal standard may be added to the extraction solution that is divided into aliquots. These aliquots are each extracted and measured by GCMS. Alternatively, aliquots of the extraction solution from a single trial could receive a standard addition. The linear response of the mass spectrometer counts per concentration of internal standard can be extrapolated to quantify the dissolved contaminant. Additionally, more than one internal standard can be used in order to span a range of molecular masses. Alternatively, standards may be used to calibrate a GCMS prior to analyzing extracted samples.

In an alternative embodiment, gas chromatography may be used for detection and/or quantitation of an analyte without using mass spectrometry. In a related embodiment, the linear trend of the peak areas of the gas chromatogram may be used for quantitation. Generally, a person of ordinary skill in the art may be able to determine the procedure and calculations to quantify and/or detect an analyte based on GCMS data.

In some embodiments, the method has a sampling rate (Rₛ) in a range of 0.005 to 0.045 liter per day (L/day) per 100 milligrams (mg) of the SGBC particles encapsulated in the membrane-biochar strip, preferably 0.01 to 0.04, preferably 0.015 to 0.035, preferably 0.02 to 0.03, or even more preferably about 0.025 L/day per 100 milligrams (mg) of the SGBC particles encapsulated in the membrane-biochar strip. Other ranges are also possible.

### EXAMPLES

The following examples demonstrate a membrane-biochar strip for water sampling described herein. The examples are provided solely for illustration and are not to be construed as limitations of the present disclosure, as many variations thereof are possible without departing from the spirit and scope of the present disclosure.

### Example 1: Preparation of Biochar

Biomass of the seagrass Halodule Uninervis (SG) were gathered from the Saudi Arabian shore to make the adsorbent. The recovered biomass was washed several times with distilled water to remove impurities and water-soluble components. The cleaned biomass was next dried at 70° C. for 24 h, followed by 4 h of thermal treatment in a tube furnace at 500° C. with a nitrogen flow (40 mL min-1). The resultant seagrass biochar (SGBC) was once more cleaned with deionized water before being overnight dried at 100° C. FIG. 1B shows the surface morphology of the prepared biochar, which is characterized using a Scanning Electron Microscope (SEM). The resultant biochar (SGBC) has a porous structure with grooves on its surface. This results from the decomposition and volatilization of the raw Halodule Uninervis seagrass material. During carbonization, the surface of the seagrass develops fractures and grooves as volatile particles and generated gases flow from the raw material swiftly over a short period.

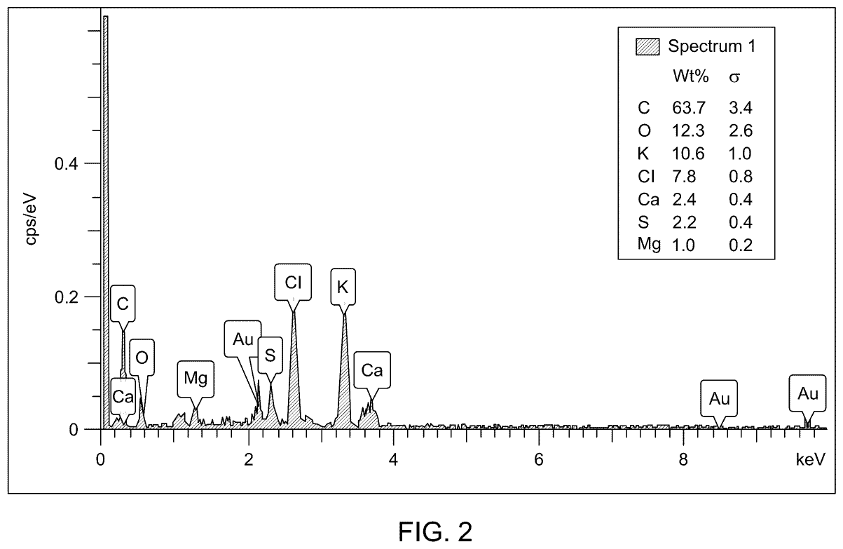

The elemental composition of the biochar's surface was obtained using the Energy Dispersive X-ray (EDX). FIG. 2 displays EDX findings. Carbon (63.7%) and oxygen (12.3%) were discovered to be present on the surface of the SGBC. Besides, the carbonized seagrass also has a low percentage of K (10.6%), Cl (7.8%), Ca (2.4%), S (2.2%), and Mg (1.0%). This shows that organic molecules with functional groups containing oxygen, such as hydroxyl and carboxylic acids, may be the source of the carbon and oxygen that make up the skeleton of the SGBC adsorbent.

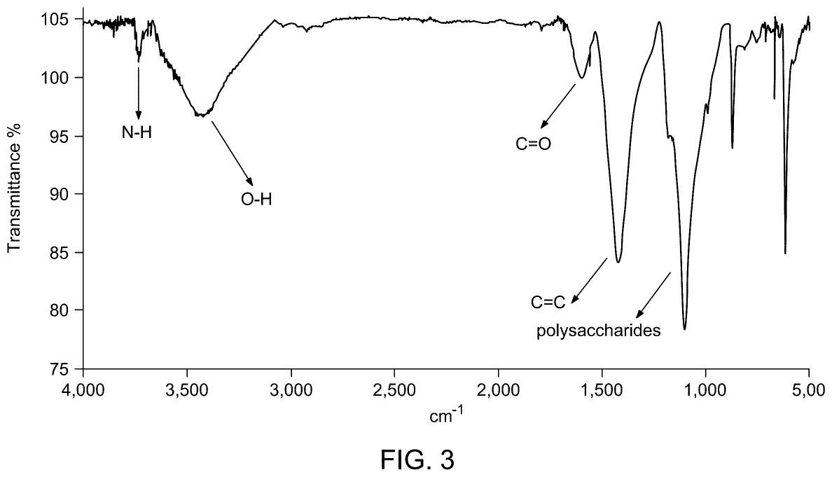

Fourier transform infrared (FT-IR) spectra of the SGBC adsorbent are displayed in FIG. 3. The presence of O—H, or the alcohol and carboxylic acid groups, is shown by the presence of the absorption stretching band at 3407-centimeter inverse (cm-1). Strong stretching peaks between 1550 and 1710 cm⁻¹ show the existence of C═O and C═C from a conjugated ketone, whereas the peak at 2921 cm⁻¹ reflects the stretching of CH₂ and CH₃ bonds linked with alkane. Additionally, polysaccharides may be responsible for stretching peaks at about 1050 cm⁻¹.

The pore volume and surface area of the prepared biochar were measured using an automated gas sorption analyzer (AutosorbiQ Quantachrome USA). The surface area and pore volume of the SGBC are 60.4 meters square per gram (m² g⁻¹) and 0.082 cubic centimeters per gram (cc g⁻¹), respectively.

### Example 2: Preparation of the Membrane-Biochar Strip

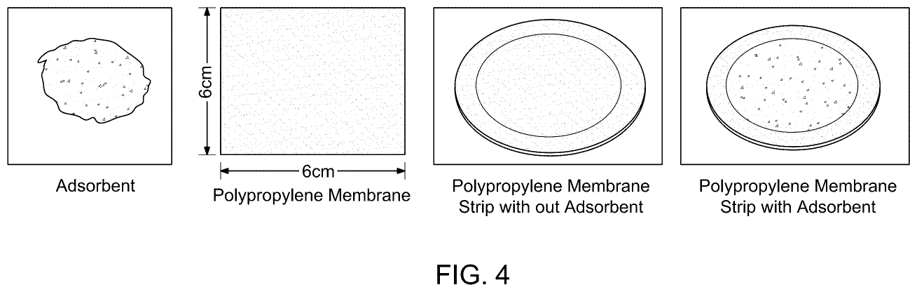

As presented in FIG. 4, SGBC adsorbent (e.g., preferably about 100 mg) was sandwiched between 6×6 cm polypropylene (PP) membrane sheets (Pore size of 100 μm), which were heat sealed using heated glass pipe at 120° C. to obtain a sampling stripe with a diameter of 4 cm. The 250 μm particle size of SGBC made it possible to maintain the adsorbent within the membrane sheets, allowing direct contact with water into the sampling matrix. Other membrane types, such as polyethersulfone (PES) and low-density polyethylene (LDPE), can make the developed strip more applicable with the integrated passive sampling technique for polar compounds and non-polar contaminants, respectively.

### Example 3: Strip Calibration and Sampling Rate Measurements

After determining the sealing temperature and ensuring the strips ruggedness, the sampling of phenols (6 phenolic compounds listed in Table 1) from the water was investigated. The sampling experiments were conducted in the laboratory following static calibration mode using glass bottles (1 L) filled with phenols-contaminated water. All experiments were performed at room temperature at about 21° C. Quality control samples were included to ensure the accuracy of the measurements: a blank water sample having a sampling strip, a sample having 10 mg/L of phenols mixture spiked in water, and two samples having 10 mg/L of phenols contaminated water and sampling strip. Then, all the bottles were agitated at 130 rotations per minute (rpm) using a shaker for five days. The solutions inside the bottles were changed every day with a new one.

TABLE 1
List of tested phenolic compounds

| Compound | Concentration (mg/L) |
|---|---|
| Phenol | 10.0 |
| 2-Chlorophenol (2-CP) | 10.0 |
| 2-Methyl phenol (2-MP) | 10.0 |
| 2,4-Dimethyl phenol (2,4-DMP) | 10.0 |
| 2,4-Dichlorophenol (2,4-DCP) | 10.0 |
| 2-Nitro phenol (2-NP) | 10.0 |

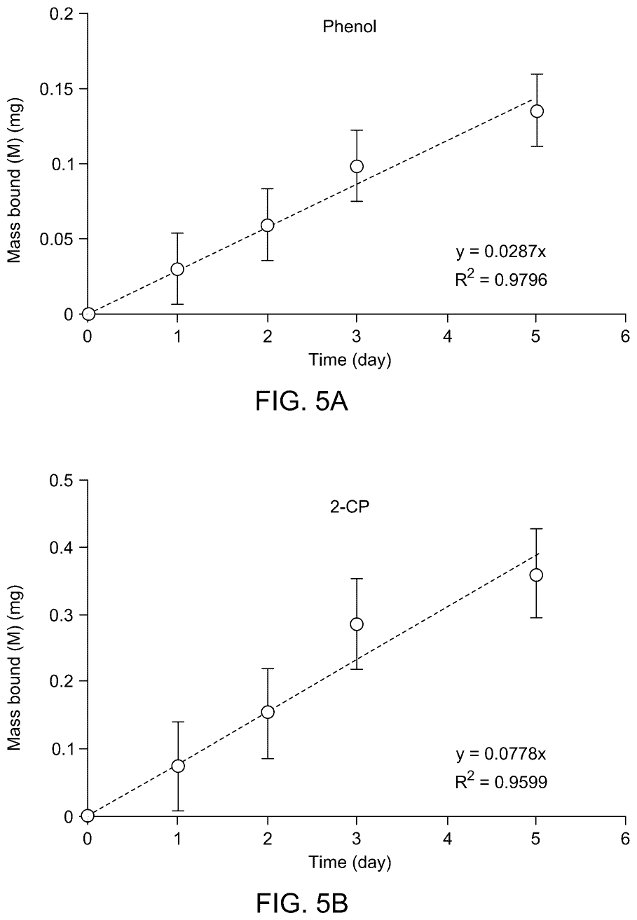

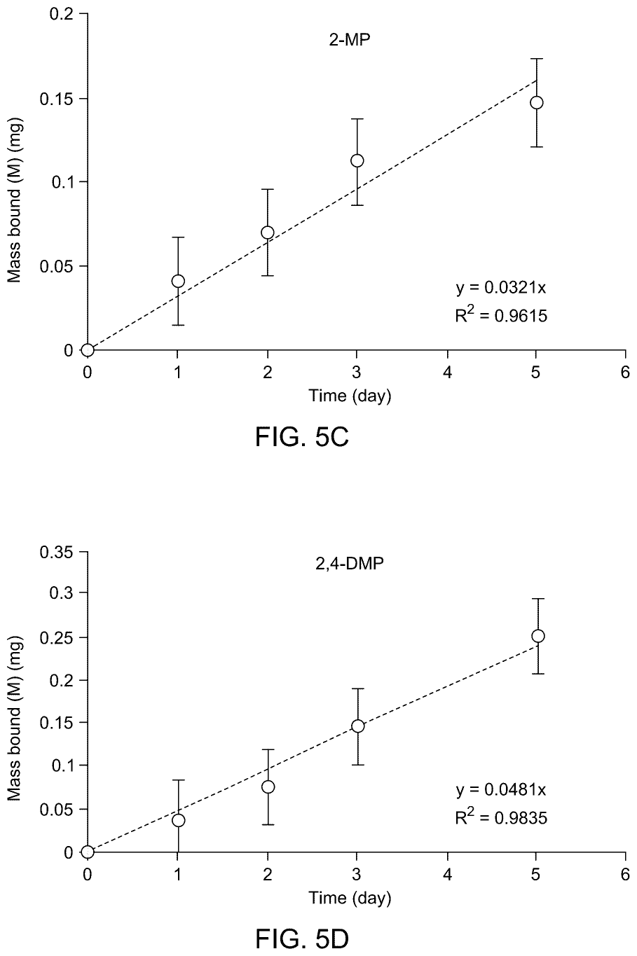

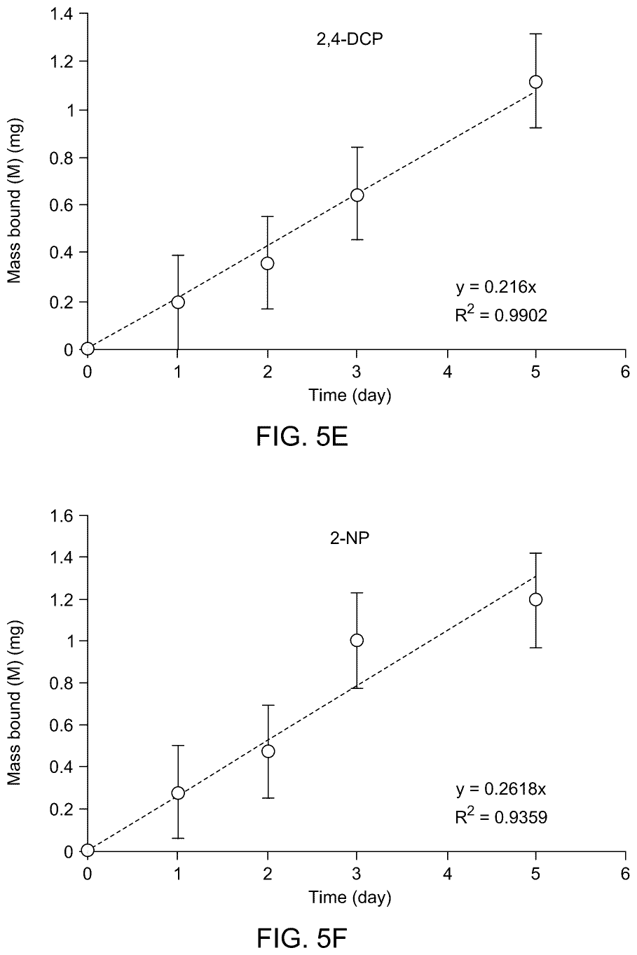

The results showed that the polypropylene membrane (strip without SGBC adsorbent) does not adsorb phenolic compounds. Besides, the phenolic compounds uptake in the developed sampling strips can be divided into two main steps: the first is a linear uptake kinetic, and the second is an equilibrium state that is not reached during sampling time (e.g., preferably about 5 days). The phenolic compounds contaminants accumulation rate is approximately linear during the first part of the uptake period (FIGS. 5A-5F), also known as the integrative regime, meaning that their mass in the adsorbent is dependent on their concentrations in the water sampled and the exposure time, as shown in equation (1):

$$M = {C}_{W} \times {R}_{S} \times t \qquad ( 1 )$$

where M is the mass of phenolic compound (mg) accumulated on the SGBC adsorbent, C_{w} the phenolic compounds concentration (mg/L) in water, Rₛ the sampling rate of the strip (L day-1), and t is the exposure time (days). Rₛ is specific for each compound (depending on their affinity on the adsorbent) and corresponds to the amount of water sampled per day. In fact, the initial quantity of phenolic compounds in the developed SGBC adsorbent (strip) is zero or negligible, and the sampling rate can be determined from the slope of the linear plot of M vs t, equation (1). Besides, the sampling rate can be determined suing equation (2):

$$Rs = M / {C}_{W} \times t \qquad ( 2 )$$

Time-weighted averaged concentrations (C_{TWA}) of phenolic compounds contaminants in water are calculated using equation (3):

$${C}_{TWA} = {C}_{s} \times {M}_{s} / Rs \times t \qquad ( 3 )$$

where C_{TWA} is the mean concentration of the phenolic compounds contaminant in the water during the deployment period (mg/L), Cₛ is the concentration in the strip (mg/g), Mₛ is the mass of SGBC adsorbent in the sampling strip (g), Rₛ is the sampling rate (L/day), which corresponds to the volume of water purified per unit of time, and t is the total exposure time (days).

The phenolic compounds concentrations in the aqueous solution were monitored by collecting 5 mL water samples and the strip after certain time intervals (every day). Prior to extraction, all the strips were washed with DI water and dried under fume hood. The phenolic compounds were extracted from the collected aqueous solutions and strips with 5 mL DCM and shaking for 15 minutes. Later 1 mL of DCM was transferred to GC vail and used for phenolic compounds analysis using gas chromatography (Agilent 7890B) coupled with FID detector.

Without accurate determination, the adsorption capacity of the biochar derived from SG as an adsorbent at the experimental conditions were found as 2.5, 7.0, 5.0, 4.5, 8.6, and 8.9 mg/g for Phenol, 2-CP, 2-MP, 2,4-DMP, 2,4-DCP, and 2-NP, respectively. After applying the aforementioned equations (1-3), the calculated sampling rate at about 21° C. for the tested phenolic compounds and the calculated time weight average concentrations of these compounds in water after 5 days are listed in Table 2.

TABLE 2
Sampling rates (Rs) of the phenolic compounds using the developed sampling strip

| | Phenol | 2-CP | 2-MP | 2,4-DMP | 2,4-DCP | 2-NP |
|---|---|---|---|---|---|---|
| Rs | 0.00545 | 0.01469 | 0.00609 | 0.00912 | 0.03788 | 0.04265 |
| (L/day) | | | | | | |
| C_{TWA} ± SD | 10.41 ± 0.81 | 10.21 ± 1.38 | 11.19 ± 1.57 | 9.12 ± 1.34 | 9.46 ± 0.83 | 10.55 ± 1.61 |
| (mg/L) | | | | | | |

It has been found the order of the sampling rate for phenolic compounds as follows: 2-NP>2,4-DCP>2-CP>2-MP>2,4-DMP>Phenol. This may be explained by considering main two factors; the electron withdrawn/donating strength of the substituent as well as the hydrophobicity of the tested phenolic compounds. For the calculated time weight average concentrations (C_{TWA}) of these six phenolic compounds, it has been found C_{TWA} values are close to the spiked concentration (10 mg/L) in the tested water. Such Rₛ values and accuracy in determining C_{TWA} signify that the developed sampling strip consisting of the SGBC adsorbent and PP membrane are promising low-cost product for contaminants sampling and determination in water.

Sampling rates for these phenolic compounds are expected to be increased by increasing water current/agitation speed, salinity, and/or temperature. Besides, increasing the organic matter is expected to decrease the sampling rates.

Numerous modifications and variations of the present disclosure are possible in light of the above teachings. It is, therefore, to be understood that within the scope of the appended claims, the invention may be practiced otherwise than as specifically described herein.

1. A membrane-biochar strip for water sampling, comprising:
two porous polymeric membrane sheets having the same dimension;
wherein the two porous polymeric membrane sheets have an average pore size of about 50 to 150 micrometers (μm);
seagrass biochar (SGBC) particles disposed between two porous polymeric membrane sheets resulting in a sandwich structure;
wherein an edge of the sandwich structure is sealed, and the SGBC particles are encapsulated in the membrane-biochar strip;
wherein the SGBC particles have an average particle size of about 150 to 400 μm; and
wherein the seagrass biochar (SGBC) particles are prepared from Halodule Uninervis seagrass.

2. The membrane-biochar strip of claim 1, wherein each of the two porous polymeric membrane sheets are independently selected from the group consisting of a polypropylene (PP) membrane sheet, a polyethersulfone (PES) membrane sheet, and a low density polyethylene (LDPE) membrane sheet.

3. The membrane-biochar strip of claim 2, wherein the two porous polymeric membrane sheets are polypropylene (PP) membrane sheets.

4. The membrane-biochar strip of claim 1, wherein the two porous polymeric membrane sheets have an average pore size of about 100 μm.

5. The membrane-biochar strip of claim 1, wherein the SGBC particles have an average particle size of about 250 μm.

6. The membrane-biochar strip of claim 1, wherein the SGBC particles have a porous structure comprising a plurality of fractures and grooves, wherein the SGBC particles have a long dimension of 250 μm, a thickness of 0.5 to 5 μm, and a width of 5 to 25 μm.

7. The membrane-biochar strip of claim 1, wherein the SGBC particles have a surface area of 50 to 70 square meters per gram (m²/g).

8. The membrane-biochar strip of claim 1, wherein the SGBC particles have a pore volume of 0.05 to 0.1 cubic centimeters per gram (cm³/g).

9. The membrane-biochar strip of claim 1, wherein the SGBC particles have an absorption capacity of up to 20 milligrams (mg) of a phenolic compound per gram of the SGBC particles.

10. The membrane-biochar strip of claim 1, wherein the SGBC particles comprises 58 to 68 wt. % C, 10 to 15 wt. % 0, 5 to 15 wt. % K, 2 to 12 wt. % Cl, 1 to 5 wt. % Ca, 1 to 5 wt. % S, 0.1 to 2 wt. % Mg, each wt. % based on a total weight of the SGBC particles, as determined by energy dispersive X-ray (EDX) analysis.

11. The membrane-biochar strip of claim 10, wherein the SGBC particles comprises about 63.7 wt. % C, about 12.3 wt. % O, about 10.6 wt. % K, about 7.8 wt. % Cl, about 2.4 wt. % Ca, about 2.2 wt. % S, about 1.0 wt. % Mg, each wt. % based on the total weight of the SGBC particles, as determined by energy dispersive X-ray (EDX) analysis.

12. A water sampling method for the determination of a dissolved contaminant in an aqueous liquid, the method comprising:
immersing the membrane-biochar strip of claim 1 in the aqueous liquid comprising the dissolved contaminant thereby allowing molecules of the dissolved contaminant to pass through the two porous polymeric membrane sheets of the membrane-biochar strip and contact with the SGBC particles; and
removing the membrane-biochar strip from the aqueous liquid after the immersing and extracting the dissolved contaminant from the membrane-biochar strip with an organic solvent to produce an extraction solution comprising the dissolved contaminant molecules.

13. The method of claim 12, wherein the dissolved contaminant is present in the aqueous liquid at a concentration of 1 to 20 milligrams per liter (mg/L) of the aqueous liquid.

14. The method of claim 12, having a sampling rates (Rₛ) in a range of 0.005 to 0.045 liter per day (L/day) per 100 milligrams (mg) of the SGBC particles encapsulated in the membrane-biochar strip.

15. The method of claim 12, wherein the dissolved contaminant is at least one phenolic compound selected from the group consisting of phenol, 2-chlorophenol (2-CP), 2-methyl phenol (2-MP), 2,4-dimethyl phenol (2,4-DMP), 2,4-dichlorophenol (2,4-DCP), and 2-nitro phenol (2-NP).

16. The method of claim 12, wherein the organic solvent is at least one selected from the group consisting of benzene, cyclohexane, ethanol, methanol, acetone, ethyl acetate, dichloromethane, toluene, and diethyl ether.

17. The method of claim 12, further comprising directly injecting the extraction solution comprising the dissolved contaminant molecules into a mass spectrometer or a chromatography column for the determination of the dissolved contaminant.

18. The method of claim 12, further comprising preparing the SGBC particles by:
washing raw seagrass and heating at a temperature of about 500 degrees Celsius (° C.) in an atmosphere of an inert gas to form the SGBC in the form of particles.

19. The method of claim 18, wherein the raw seagrass comprises Zostera Marina seagrass, Posidonia Oceanica seagrass, Halodule Uninervis seagrass, Thalassia Testudinum seagrass, Syringodium Filiforme seagrass, Enhalus Acoroides seagrass, Cymodocea Nodosa seagrass, Thalassodendron Ciliatum seagrass, Phyllospadix Spp seagrass, and Posidonia Australis seagrass.

20. The method of claim 19, wherein the raw seagrass is Halodule Uninervis seagrass.
